(19) United States
(12) Patent Application Publication
(10) Pub. No.: US 2022/0276262 A1
(43) Pub. Date: Sep. 1, 2022

(54) Methods and Products for Assessing Lysosomal System Flux

(71) Applicant: SOUTH AUSTRALIAN HEALTH AND MEDICAL RESEARCH INSTITUTE LIMITED, Adelaide, South Australia (AU)

(72) Inventors: Timothy John Sargeant, Grange, South Australia (AU); Julien Michel Bensalem, Woodville South, South Australia (AU)

(21) Appl. No.: 17/637,494

(22) PCT Filed: Aug. 28, 2020
(86) PCT No.: PCT/AU2020/050908

§ 371 Date: Feb. 23, 2022

(30) Foreign Application Priority Data
Aug. 30, 2019 (AU) 2019903187
Dec. 19, 2019 (AU) 2019904822

(51) Int. Cl. G01N 33/68; G01N 1/34

(52) U.S. Cl. CPC G01N 33/6893; G01N 1/34

(57) ABSTRACT

The present disclosure relates to relates to methods and products for assessing lysosomal system flux. In addition, the present disclosure also provides systems for assessing lysosomal system flux, and methods of identifying markers indicative of lysosomal system flux. In certain embodiments, the present disclosure provides a method of assessing lysosomal system flux in a subject. The method comprises determining the level of a lysosomal system marker in a sample of whole blood from the subject, the level of the lysosomal system marker being determined based on the level of the marker following treatment of the whole blood with an inhibitor of lysosomal system function.

### PRIORITY CLAIM

This application claims priority to both Australian Provisional Patent Application 2019903187 filed on 30 Aug. 2019 and Australian Provisional Patent Application 2019904822 filed on 19 Dec. 2019, the contents of which are hereby incorporated by reference.

### FIELD

The present disclosure relates to methods and products for assessing lysosomal system flux. In addition, the present disclosure provides systems for assessing lysosomal system flux, and methods of identifying markers indicative of lysosomal system flux.

### BACKGROUND

Autophagic flux (referred to also as “lysosomal system flux”) is the acquisition, transport, and degradation of unwanted or damaged material in the lysosomal system. The efficient execution of this entire series of events is important for two aspects of physiology. The first is cellular quality control that is critical for healthy tissue function. The second major function of autophagic flux is nutrient recycling to adapt to starvation.

Accumulating human and preclinical research shows that inefficient autophagic flux plays a major and direct role in prevalent diseases such as dementia, and heart disease. Further, lysosomal system function supports healthy proteostasis, the dysfunction of which is a hallmark of aging.

As such, modification of lysosomal system function is important to health, and interventions that modify lysosomal system function (including nutrition, exercise, or pharmacological agents) are likely to be translated into clinical practice.

To date no direct methods that directly measure autophagic flux in human samples have been developed, thus providing a barrier to translating interventions that target autophagy. Many studies have measured lysosomal system proteins in human samples, which have been used as a proxy for autophagic flux. These studies do not measure flux of material through the lysosomal system and are not a reliable measure of lysosomal system activity.

The gold standard test for assessing autophagic flux is western blot for an LC3 protein isoform without and with inhibition of lysosomal proteolysis. This technique is commonly applied to cells in culture. However, this method has not been adapted successfully to organotypic human samples that reflect both the nutritional and endocrine status of an individual, both factors which directly impact mTOR signalling and thus lysosomal system function.

As such, it is not known what kinds of variation impact autophagic flux in a human population, or what important co-variates might look like. Further, because lysosomal flux has not been measured in humans, autophagic flux cannot be used as a primary endpoint in itself for clinical trials. In the absence of such a measure, disease-specific endpoints would have to be used and the impact of treatments that aim to boost autophagic flux will remain unclear. This gap in knowledge represents an urgent unmet need that is currently hampering translation of a wealth of data on the lysosomal system that already exists in the scientific literature.

Accordingly, there is a need to be able to measure autophagic flux in a manner that reflects the autophagic flux in a subject.

### SUMMARY

The present disclosure relates to methods and products for assessing lysosomal system flux. In addition, the present disclosure provides systems for assessing lysosomal system flux, and methods of identifying markers indicative of lysosomal system flux.

Certain embodiments of the present disclosure provide a method of assessing lysosomal system flux in a subject, the method comprising determining the level of a lysosomal system marker in a sample of whole blood from the subject, the level of the lysosomal system marker being determined based on the level of the marker following treatment of the whole blood with an inhibitor of lysosomal system function.

Certain embodiments of the present disclosure provide a method of assessing lysosomal system flux in a subject, the method comprising:
obtaining a sample of whole blood from the subject;
treating the sample of whole blood with an inhibitor of lysosomal system function; and
determining the level of a lysosomal system marker in the whole blood so treated as compared to the level of the lysosomal system marker in whole blood without treatment.

Certain embodiments of the present disclosure provide use of a lysosomal system marker in whole blood treated with an inhibitor of lysosomal system function to determine the level of lysosomal system flux in the subject.

Certain embodiments of the present disclosure provide a kit for assessing lysosomal system flux in whole blood, the kit comprising the following components:
a reagent for detecting a lysosomal system marker; and
optionally one or more of an inhibitor of lysosomal system function, an anti-coagulant,
a biochemical extraction reagent, and a cell lysis reagent.

Certain embodiments of the present disclosure provide a system for assessing lysosomal system flux in a subject, the system comprising:
a processor for receiving data indicative of the level of a lysosomal system marker in whole blood treated with an inhibitor of lysosomal system function; and
a memory with software resident in the memory, and accessible to the processor, wherein the software comprises a series of instructions executable by the processor to convert the data to a measurement of lysosomal system flux in the subject.

Certain embodiments of the present disclosure provide a method of treating a subject suffering from, or susceptible to, a disease, condition or state associated with autophagic dysfunction, the method comprising determining the lysosomal system flux in the subject by a method as described herein and treating the subject on the basis of the level of lysosomal system flux determined.

Certain embodiments of the present disclosure provide a method of identifying a marker present in blood indicative of lysosomal system flux in a subject, the method comprising:
determining the level of a candidate marker indicative of lysosomal system flux in whole blood treated with an inhibitor of lysosomal system function; and
identifying the candidate marker as a marker indicative of lysosomal system flux.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

For a better understanding of the present disclosure, and to show more clearly how the present disclosure may be carried into effect according to one or more embodiments thereof, reference will be made, by way of example, to the accompanying figures.

FIG. 1 shows titration of chloroquine into whole blood and analysis of LC3B protein. (A) Chloroquine was titrated into whole blood samples at final concentrations ranging from 0-200 μM. PBMCs were isolated from the whole blood after incubation at 37° C. and LC3B-II was analysed by western blot. Results were quantified and plotted for four individual people (lines S1-S4) or displayed as the mean±SEM for four people (closed circles). (B) Western blots showing LC3B-II and two loading controls (β-actin and GAPDH).

FIG. 2 shows titration of chloroquine into whole blood and analysis of P62 protein. (A) Chloroquine was titrated into whole blood samples at final concentrations ranging from 0-200 μM. PBMCs were isolated from the whole blood after incubation at 37° C. and P62 was analysed by western blot. Western blots show P62 and two loading controls (β-actin and GAPDH).

FIG. 3 shows addition of rapamycin to whole blood ex-vivo enhances autophagic flux in PBMCs. Rapamycin or vehicle (DMSO) was added to whole blood for the one-hour incubation at 37° C. (A) Quantitative data were derived from western blots of LC3BII. (B) Samples were blotted for LC3B and β-actin was used as a loading control. (C) ΔLC3BII ([+CQ]LC3BII−[−CQ]LC3BII=ΔLC3BII) was derived and showed an increase in autophagic flux for the samples incubated with rapamycin.

FIG. 4 shows assessment of experimenter induced variation. Variation in the test was measured by distributing blood from three different subjects (S1-S3) each to three different scientists. Variation within the subjects as measured by three different people is smaller than variation between different subjects.

FIG. 5 shows autophagic flux can be measured in primary culture of human leukocytes in vitro. (A) Human blood was fractionated using Lymphoprep, and PBMCs and PMNs were cultured without and with chloroquine in RPMI. (B) Cell populations derived from Lymphoprep fractionation were determined using flow cytometry. (C) Western blots for lysosomal system cargos (LC3B and P62) across PBMCs and PMNs are shown (N=3).

FIG. 6 shows autophagic flux in PBMCs can be measured by lysosomal inhibition in whole human blood. (A) Chloroquine was titrated into whole blood samples at final concentrations ranging from 0-200 μM and incubated at 37° C. for 1 h before PBMCs were extracted for analysis of LC3B-II by western blot. (B) Western blot for LC3B from PBMCs that were exposed to chloroquine while in whole blood. (C) Blood from four subjects was processed according to the diagram in (A) and is displayed as ΔLC3B-II normalized to β-ACTIN. (D) ATG5 KO HeLa cells that cannot lipidate LC3B-I to form LC3B-II were used to determine that the LC3B antibody used in this study was specific.

FIG. 7 shows measurement of autophagic flux in whole blood is repeatable and replicable. (A) To determine if measurement of autophagic flux in PBMCs were repeatable when processed in parallel, two vials of blood from the same subject were given to one scientist to process. (B) Western blot analysis of LC3B. (C) ΔLC3-II normalized to β-ACTIN from four subjects is shown. (D) To determine stability of autophagic flux measurement blood was collected from the same fasted subject on two consecutive days. (E) Samples were analyzed for LC3B by western blot. (F) ΔLC3B-II normalized to β-ACTIN is shown for two consecutive days for three subjects. (G) To determine replicability of autophagic flux measurement, two vials of whole blood were taken from subjects, pooled, and split between three different scientists to independently process. (H) LC3B was analyzed by western blot. (I) ΔLC3B-II normalized to β-ACTIN is shown, as determined by three different scientists working in parallel on blood from three subjects.

FIG. 8 shows measurement of autophagic flux in different collection conditions—storage of whole blood and collection tube type. (A) Whole blood was taken from subjects and stored at room temperature for four-hours and then processed for measurement of autophagic flux by western blot for LC3B. (B) LC3B-II normalized using β-ACTIN was determined for blood taken from three subjects and processed for autophagic flux after storage at room temperature (RT) for 0 h or 4 h. (C) Whole blood was taken from donors and stored on ice for four-hours and then processed for measurement of autophagic flux by western blot for LC3B. (D) LC3B-II normalized using β-ACTIN was determined for blood taken from three subjects and processed for autophagic flux after storage on ice for 0 h or 4 h. (E) Whole blood was collected from subjects in different tubes—lithium-heparin- (Li-Hep) or EDTA-containing tubes were tested. Blood was then processed for analysis of autophagic flux by western blot for LC3B. (F). LC3B-II normalized using β-ACTIN was determined for blood taken from three subjects using lithium-heparin- (Li-Hep) or EDTA-containing tubes.

FIG. 9 shows addition of leucine and insulin to whole blood reduces autophagic flux. (A) Whole blood was taken and incubated without or with a mixture of leucine and insulin for three-hours to mimic the effect of a protein-rich meal. Whole blood was then processed for measurement of autophagic flux. (B) Western blots showing analysis of LC3B in blood cultured without or with leucine and insulin. (C) ΔLC3B-II normalized using β-ACTIN was determined for blood taken from n=10 subjects. The effect of leucine and insulin was statistically significant, P=0.0145, paired t-test.

FIG. 10 shows that bafilomycin in an ethanol vehicle is capable of stopping autophagic flux when added to whole blood.

FIG. 11 shows that the time course analysis of chloroquine incubation in whole blood.

### DETAILED DESCRIPTION

The present disclosure relates to methods and products for assessing lysosomal system flux. In addition, the present disclosure provides systems for assessing lysosomal system flux, use of the methods for informing treatment of a subject suffering from, or susceptible to, a disease, condition or state associated with lysosomal system dysfunction, and methods of identifying markers indicative of lysosomal system flux.

The present disclosure is based, at least in part, on the quantitative measurement of autophagic flux for the first time in an organotypic human sample that maintains the nutritional and signalling status inherent to the individual. In the present studies, the autophagic flux was measured in peripheral blood mononuclear cells (PBMCs) while the cells still existed in whole human blood to preserve the individual-specific nutritional and cell signalling environment, and indicates that measurement of autophagic flux in whole bloods reflects the status in the human body.

Certain embodiments of the present disclosure provide a method of assessing lysosomal flux in a subject.

This embodiment of the present disclosure permits the determining of lysosomal system flux in a subject, using a marker that is indicative of the flux in the system on whole blood.

In this regard, it will be appreciated that the marker may be a marker directly associated with the flux of the lysosomal system, or alternatively may be a marker that is a proxy marker for the flux of the lysosomal system.

In certain embodiments, the present disclosure provides a method of assessing lysosomal system flux in a subject, the method comprising determining the level of a lysosomal system marker in whole blood from the subject.

In certain embodiments, the method of assessing lysosomal system flux in a subject comprises determining the level of a lysosomal system marker in a sample of whole blood from the subject.

In certain embodiments, the method comprises treating a sample of whole blood with an inhibitor of lysosomal system function. In certain embodiments, the level of the lysosomal system marker is determined based on the level of the marker in a sample of whole blood treated with an inhibitor of lysosomal system function.

In certain embodiments, the present disclosure provides a method of assessing lysosomal system flux in a subject, the method comprising determining the level of a lysosomal system marker in a sample of whole blood from the subject, the level of the lysosomal system marker being determined based on the level of the marker following treatment of the whole blood with an inhibitor of lysosomal system function.

The term “lysosomal system flux” as used herein refers to the activity of the lysosomal system. The lysosomal system is a series of organelles in the endocytic and autophagic pathways where various cargo molecules required for normal cellular function are internalized, sequestered, and recycled. The lysosomal system includes early endosomes, recycling endosomes, late endosomes, the lysosome, and autophagosomes which delivers intracellular contents to the lysosome. Maturation of endosomes and/or autophagosomes into a lysosome, or fusion with a lysosome creates an acidic environment within the cell for proteolysis and recycling of various cellular components. The activity of the system is likely influenced by individual genetic variation (such as genetic variation in lysosomal system genes that is known to associate with Alzheimer's disease). It is also acutely impacted by changing physiological conditions that include the level and/or location of the various functions of the system, and includes, but is not limited to, activation of receptor signalling by ligands such as epidermal growth factor and insulin, or the activation of autophagy by calorie or protein restriction through activation of AMPK and the inhibition of mTOR. As such, lysosomal system function is also highly likely to be impacted by nutrition-related disorders in humans such as obesity and diabetes.

In certain embodiments, the subject is a human subject, although it will be appreciated that veterinary and research applications of the present disclosure in animals are also contemplated.

In certain embodiments, the subject is an adult human subject.

In certain embodiments, the subject is a paediatric subject or a neonatal subject.

In certain embodiments, the subject is suffering from, or susceptible to, a disease, condition or state associated with lysosomal system dysfunction.

In certain embodiments, the subject is a subject for which assessment of lysosomal system flux provides information as to the state of the subject. In certain embodiments, the subject is a subject for which information on lysosomal system flux is required for diagnostic or prognostic purposes. In certain embodiments, the subject is a subject for which information on lysosomal system flux is required for treatment purposes.

Diagnostic and prognostic applications of the present disclosure are contemplated. In certain embodiments, a method as described herein is used for diagnostic or prognostic purposes.

A suitable amount of blood may be used for analysis in the present disclosure.

In this regard, typically about 6 ml of blood is used, which is split in two 3 ml samples, one 3 ml sample without the inhibitor and one 3 ml sample with the inhibitor (eg chloroquine, bafolimycin). However, it will be appreciated that the amount of blood used for analysis may be reduced. It is envisaged that an amount of 1 ml or less of blood may be suitable, particularly when testing children or babies. In this regard, experiments have been conducted that confirm that 500 μL samples of whole blood contain sufficient protein to conduct the assay.

In certain embodiments, the method of the present disclosure comprises treating the sample of whole blood with suitable amount of an inhibitor of lysosomal system function.

In certain embodiments, the inhibitor of lysosomal system function comprises one or more of chloroquine, bafilomycin A1, E-64d, leupeptin, and pepstatin A. Such agents are known in the art and may be obtained commercially or obtained by a method known in the art. Other inhibitors are contemplated. Methods for determining the ability of an agent to act as an inhibitor of lysosomal system function are known in the art.

In certain embodiments, the inhibitor of lysosomal system function comprises chloroquine and/or bafilomycin A1.

A suitable concentration of the inhibitor to be used for treating whole blood may be selected.

In certain embodiments, the inhibitor of lysosomal system function comprises chloroquine. In certain embodiments, the concentration of chloroquine to be used in treating whole blood is in the range from 10 μM to 300 μM. In certain embodiments, the concentration of chloroquine to be used in treating whole blood is in the range from one of 10 μM to 250 μM, 10 μM to 200 μM, 50 μM to 300 μM, 50 μM to 250 μM, or 50 μM to 200 μM. Other ranges are contemplated.

In certain embodiments, the inhibitor of lysosomal system function comprises bafilomycin. In certain embodiments, the concentration of bafilomycin to be used in treating whole blood is in the range from 50 nM to 800 nM. In certain embodiments, the concentration of bafilomycin to be used in treating whole blood is in the range from one of 50 nM to 800 nM, 50 nM to 500 nM, 50 nM to 400 nM, 50 nM to 300 nM, 50 nM to 200 nM, or 50 nM to 200 nM. Other ranges are contemplated.

In certain embodiments, the whole blood sample is treated with the inhibitor of lysosomal system function for a time of 2 hours or less, 90 minutes or less, 60 minutes or less, 45 minutes or less, 30 minutes or less, or 15 minutes or less.

In certain embodiments, the whole blood sample is treated with the inhibitor of lysosomal system function for a time of at least 15 minutes, at least 30 minutes, at least 45 minutes, at least 60 minutes, at least 90 minutes or at least 2 hours.

In certain embodiments, the whole blood sample is treated with the inhibitor of lysosomal system function for a period of time of time ranging from 15 minutes to 2 hours, 30 minutes to 2 hours, 45 minutes to 2 hours, 60 minutes to 2 hours, 90 minutes to 2 hours, 15 minutes to 90 minutes, 30 minutes to 90 minutes, 45 minutes to 90 minutes, 60 minutes to 90 minutes, 15 minutes to 60 minutes, 30 minutes to 60 minutes, 15 minutes to 45 minutes, 30 minutes to 45 minutes, or 15 to 30 minutes.

Methods for obtaining samples from blood are known in the art. Samples obtained from a subject may be processed as described herein.

In certain embodiments, the sample of whole blood is exposed to, or treated with, the inhibitor of lysosomal system function as soon as it is collected.

In certain embodiments, the sample of whole blood is stored at reduced temperature, such as 4° C. or below, for 4 hours or less, 3 hours or less, 2 hours or less, 1 hour or less, or 30 minutes or less before being treated with the inhibitor of lysosomal system function.

In certain embodiments, the sample of blood is placed into a collection container already containing the inhibitor of lysosomal system function at the time of sampling.

In certain embodiments, the sample of whole blood is treated with an anti-coagulant. In certain embodiments, the sample of whole blood is treated with lithium heparin. In certain embodiments, the sample of whole blood is treated with EDTA.

In certain embodiments, the sample of whole blood treated with the inhibitor of lysosomal system function is analysed as soon as practical after treatment with the inhibitor. In certain embodiments, the sample of whole blood is analysed immediately after treatment.

In certain embodiments, the sample of whole blood treated with the inhibitor of lysosomal system function is analysed after holding the sample at a reduced temperature, such as 4° C. or below. In certain embodiments, the sample of whole blood after treatment is analysed after 1 hour or less, 2 hours or less, 3 hours or less, or 4 hours or less.

In certain embodiments, the sample, and/or cells contained in the whole blood sample therein, are further processed to allow detection of the lysosomal system marker. Methods for further processing a blood sample, and/or cells within a blood sample, are known in the art. In certain embodiments, the method comprises isolation or enrichment of peripheral blood mononuclear cells, polymorphonuclear cells or total white blood cells, and the determination of the level of the marker in the cells.

For example, in the case of total white blood cells a sample of whole blood may be obtained, red blood cells removed by a standard technique, total white cells isolated (eg by centrifugation) and the pellet analysed for the level of the lysosomal system flux marker.

In certain embodiments, the sample of whole blood and/or cells contained therein are further processed to allow detection of the lysosomal system marker.

In certain embodiments, the assessing of the lysosomal flux comprises detecting the lysosomal system marker. In certain embodiments, the assessing of the lysosomal flux comprises measuring the level of the lysosomal system marker. In certain embodiments, the assessing of the lysosomal flux comprises determining the level of the lysosomal system marker. Examples of methods for detecting and assessing the level of a marker include immunological detection methods, methods assessing the level of expression of the marker, such as RNA analysis, RT-PCR, protein analysis, flow cytometric levels, immunocytochemical detection and analysis by microscopy, and transmission electron microscopy, all of which are known in the art.

In certain embodiments, the method comprises determining the level of the lysosomal system marker using immunological detection. In certain embodiments, the immunological detection comprises ELISA. In certain embodiments, the immunological detection comprises Western blotting. Antibodies to the specific target protein may be obtained commercial or produced by a method known in the art. Immunological detection methods are described, for example, in “Assay Guidance Manual” Sittampalam G S, Grossman A, Brimacombe K, et al., editors. Bethesda (Md.): Eli Lilly & Company and the National Center for Advancing Translational Sciences; 2004-.

In this regard, for ELISA for detection and analysis of LC3B-II, it will be appreciated that typically a further reagent is used in conjunction with the regular reagents of an ELISA kit, namely a saponin or a saponin-like detergent to remove soluble LC3B-I before use of a lysis reagent for homogenisation and analysis of the LC3B-II, which is known in the art.

Methods for performing Western blotting or immunosorbent assays are known in the art, for example as described in Sambrook et al. Molecular Cloning: A Laboratory Manual (4th ed., Cold Spring Harbor Laboratory Press, Cold Spring Harbor, N.Y. (2012) and Ausubel et al Current Protocols in Molecular Biology (2012) John Wiley & Sons.

In certain embodiments, the method comprises determining the level of the lysosomal system marker in one or more of peripheral blood mononuclear cells, derivative cellular populations, and polymorphonuclear cells (PMNs) derived from the whole blood treated with the inhibitor of lysosomal system function.

In certain embodiments, the lysosomal system marker comprises a protein marker. Methods for detecting protein markers are known in the art. In certain embodiments, detection of a protein marker comprises immunological detection. Antibodies (and antigenic parts thereof) for use in immunological detection may be obtained commercially or produced by a method known in the art.

In certain embodiments, the lysosomal system marker comprises an RNA. Methods for detecting RNA markers are known in the art, such as Northern analysis, RNase protection and RT-PCR. In certain embodiments, detection of an RNA marker comprises RT-PCR detection. Primers for use with a reverse transcriptase and for PCR detection may be obtained commercially or produced by a method known in the art.

In certain embodiments, the lysosomal system marker comprises a lipid. Methods for detecting lipids are known in the art. Other types of lysosomal system markers are known in the art, such as small molecules.

In certain embodiments, the lysosomal system marker comprises an LC3 protein and/or a GABARAP/GATE-16 protein, and/or an LC3 interacting cargo adaptor protein. LC3 protein refers to one of three Microtubule-associated proteins 1A/1B light chain 3: MAP1LC3A (LC3A, UniProtKB-Q9H492), or MAP1LC3B (LC3B, UniProtKB: Q9GZQ8), or MAP1LC3C (LC3C, UniProtKB-Q9BXW4). The GABARAP/GATE-16 proteins (Gamma-aminobutyric acid receptor-associated protein) in humans comprises of GABARAP (UniProtKB-O95166), GABARAPL1 (UniProtKB-Q9H0R8), and GABARAPL2 (UniProtKB-P60520). In addition, cargo receptor proteins that interact with LC3 and/or GABARAP/GATE-16 proteins may also be used, including but not limited to SQSTM1 (P62, UniProtKB-Q13501), NBR1 (UniProtKB-Q14596) or OPTN (UniProtKB-Q96CV9). Orthologues in other species may be identified by a method known in the art. Methods for detecting the aforementioned proteins are known in the art.

In certain embodiments, the lysosomal system marker comprises a LC3B protein. (UniProt/Swiss-Prot: Q9GZQ8).

In certain embodiments, the lysosomal system marker comprises LC3B-II protein (UniProt/Swiss-Prot: Q9GZQ8), which is a lipidated isoform of LC3B.

In certain embodiments, the lysosomal system marker comprises P62 protein (UniProt/Swiss-Prot: Q13501).

In certain embodiments, the method comprises use of a device.

In certain embodiments, the device comprises a device for determining the level of a lysosomal system marker in a sample of cells and/or lysed cells derived from a whole blood treated with an inhibitor of lysosomal system function.

In certain embodiments, the device comprises a device for separating PBMC from whole blood.

In certain embodiments, the device comprises a device for processing whole blood and/or cells contained therein for detecting and determining a lysosomal system markers.

In certain embodiments, the method comprises comparison of the level of the lysosomal system marker with the level of a marker in whole blood without treatment with an inhibitor of lysosomal system function. In certain embodiments, the method comprises comparison of the level of the lysosomal system marker with the level of the same marker in whole blood without treatment with an inhibitor of lysosomal system function. In certain embodiments, the method comprises comparison of the level of the lysosomal system marker before and after treatment with an inhibitor of lysosomal system function. In certain embodiments, the method comprises comparison of the level of the lysosomal system marker with the level of a different marker in whole blood without treatment with an inhibitor of lysosomal system function. In certain embodiments, the method comprises comparison of the level of the lysosomal system marker with a known level of marker.

In certain embodiments, the method comprises receiving data on the level of the lysosomal system marker. In certain embodiments, the method comprises receiving data on the level of the lysosomal system marker from a device.

In certain embodiments, the device determines the level of a lysosomal system marker in a sample of cells and/or lysed cells derived from a whole blood treated with an inhibitor of lysosomal system function.

In certain embodiments, the method comprises comparing the level of the lysosomal system marker to the level of the marker in blood without treatment. In certain embodiments, the method comprises comparing the level of the lysosomal system marker to the level of the marker in blood from another source. In certain embodiments, the method comprises comparing the level of the lysosomal system marker to the level of the marker in blood before treatment. In certain embodiments, the method comprises comparing the level of the lysosomal system marker to a reference level of the marker and/or one or other markers. In certain embodiments, the method comprises comparing the level of the lysosomal system marker to a known or control level of the marker and/or one or other markers.

In certain embodiments, the level of the lysosomal system marker is normalised to one or more other markers, or to the total amount of sample used. For example, for RNA analysis the level of transcription of a house keeping gene, such as GAPDH, may be utilised. For proteins, normalisation can be achieved using normalisation to total protein used, or by creating a standard curve for proteins such as β-Actin, GAPDH or α-Tubulin. Computer assisted assessment of any of the aforementioned levels may be undertaken. Such data can be held on computer readable memory or in a database to assist with determining the level of a lysosomal system marker.

In certain embodiments, the method comprises computer assisted assessment.

In certain embodiments, the method comprises computer assisted assessment of the level of the lysosomal system marker. Methods for assessing the level of the marker are described herein.

In certain embodiments, the method comprises computer assisted assessment of the level of the lysosomal system marker after treatment with an inhibitor of lysosomal system function. In certain embodiments, the method comprises computer assisted assessment of the level of the lysosomal system marker before and after treatment with an inhibitor of lysosomal system function. In certain embodiments, the method comprises computer assisted assessment of the level of the lysosomal system marker as compared to a known or control level of the marker and/or one or other markers.

In certain embodiments, the computer assisted assessment comprises use of an algorithm. In certain embodiments, the algorithm provides a measure or calculation of lysosomal system flux based on the level of the lysosomal system marker determined. Algorithms for assessing lysosomal system flux may be developed by a suitable person, and may be embodied in suitable software. Information may be held on a database accessible to a computer.

In certain embodiments, the algorithm provides a measure or calculation of lysosomal flux based on the level of the lysosomal system marker determined after treatment with an inhibitor of lysosomal system function. In certain embodiments, the algorithm provides a measure or calculation of lysosomal flux based on the level of the lysosomal system marker determined before and after treatment with an inhibitor of lysosomal system function.

In certain embodiments, the computer assisted assessment involves on-line interrogation of a computer with the level of the lysosomal system marker (or data associated therewith) determined. In certain embodiments, the computer assisted assessment involves on-line interrogation of a computer with data relating to, or associated with, the level of the lysosomal system marker determined.

In certain embodiments, the computer assisted assessment involves interrogation of a computer with the level of the lysosomal system marker determined by a device. In certain embodiments, the computer assisted assessment involves on-line interrogation of a computer with the level of the lysosomal system marker determined by a device.

In certain embodiments, the method comprises using a computer processor. In certain embodiments, data associated with the performance of a method as described herein is held in computer readable memory.

In certain embodiments, the computer processor comprises instructions that when executed cause the processor to compare data associated with the level of a lysosomal system maker with data associated with the level of the marker before or without treatment, or data known to be indicative of the level of the level before or without treatment, and/or data required for normalisation of levels, and thereby provide a calculation or measure of the lysosomal system flux. In certain embodiments, the data associated with the level of the maker and/or the data known to be indicative of the level of the level before or without treatment is held in computer readable memory.

In certain embodiments, the method comprise use of a device for determining the level of a lysosomal system marker in a sample of cells and/or lysed cells derived from a whole blood treated with an inhibitor of lysosomal system function. Devices are described herein.

In certain embodiments, the present disclosure provides a method of assessing lysosomal system flux in a subject, the method comprising:
obtaining a sample of whole blood from the subject;
treating the sample of whole blood with an inhibitor of lysosomal system function; and
determining the level of a lysosomal system marker in the whole blood so treated.

In certain embodiments, the present disclosure provides a method of assessing lysosomal system flux in a subject, the method comprising:
obtaining a sample of whole blood from the subject;
treating the sample of whole blood with an inhibitor of lysosomal system function; and
determining the level of a lysosomal system marker in the whole blood so treated as compared to the level of the lysosomal system marker in whole blood without treatment.

In certain embodiments, a method as described herein may be used for diagnosis of a disease, condition or state associated with lysosomal system dysfunction. In certain embodiments, a method as described herein may be used for prognosis of a disease, condition or state associated with lysosomal system dysfunction. In certain embodiments, a method as described herein may be used for screening for a disease, condition or state associated with lysosomal system dysfunction. In certain embodiments, a method as described herein may be used to inform treatment of a subject. Other uses are contemplated.

Certain embodiments of the present disclosure provide use of a lysosomal system marker in whole blood to determine the level of lysosomal system flux in a subject, as described herein.

Certain embodiments of the present disclosure provide use of a lysosomal system marker in whole blood treated with an inhibitor of lysosomal system function to determine the level of lysosomal system flux in the subject, as described herein.

Certain embodiments of the present disclosure provide a kit for performing a method as described herein.

Certain embodiments of the present disclosure provide a kit for assessing lysosomal flux in whole blood.

In certain embodiments, the kit is used to perform a method as described herein.

In certain embodiments, the kit comprises one or more reagents or components as described herein.

In certain embodiments, the kit comprises one or more of the following components: reagent(s) for detecting a lysosomal system marker, an inhibitor of lysosomal system function as a reference or control, anti-coagulant(s), reagents for biochemical extraction (eg reagents for extraction of LC3B-I using saponin), reagents for lysis of cells (eg lysis of cells for ELISA detection of remaining LC3B-II), a collection tube, a collection tube comprising an inhibitor of lysosomal system function, and a plate or other platform for detecting the presence of the marker using an immunosorbent assay, such as an ELISA plate. Other reagents are contemplated.

In certain embodiments, the present disclosure provides a kit for assessing lysosomal system flux in whole blood, the kit comprising the following components:
a reagent for detecting a lysosomal system marker; and
optionally one or more of an inhibitor of lysosomal system function, an anti-coagulant, a biochemical extraction reagent, a cell lysis reagent, and an ELISA plated coated with an antibody, or a binding part thereof, to the lysosomal system marker.

In certain embodiments, the present disclosure provides a kit for assessing lysosomal system flux in whole blood, the kit comprising the following components:
a collection tube comprising an inhibitor of lysosomal system function; and/or
an ELISA plate coated with an antibody, or a binding part thereof, to the lysosomal system marker.

Methods and reagents for detecting lysosomal system markers are as described herein. Anti-coagulants, biochemical extraction agents (eg saponin) and cell lysis reagents are described herein and are known in the art.

Certain embodiments of the provide a method for assessing lysosomal flux in whole blood, the method comprising use of a kit as described herein.

Certain embodiments of the present disclosure provide a blood collection tube comprising an inhibitor of lysosomal system function.

Blood collection tubes are known in the art and available commercially.

Inhibitors of lysosomal system function as described herein. A suitable amount of the inhibitor may be selected, as described herein.

In certain embodiments, the tube is coated with a suitable amount of the inhibitor. In certain embodiments, the tube contains a suitable amount of the inhibitor in a solid or dried form.

In certain embodiments, the blood collection tube comprises a suitable amount of an anti-coagulant. Examples of anti-coagulants include EDTA, sodium citrate, CTAD, lithium/sodium heparin, sodium fluoride, acid citrate dextrose, and sodium polyanethol sulfonate.

Certain embodiments of the present disclosure provide use of collection tube as described herein in a method or kit as described herein.

Certain embodiments of the present disclosure provide a system for assessing lysosomal system flux in a subject.

In certain embodiments, the present disclosure provides a system for assessing lysosomal system flux in a subject, the system comprising:
a processor for receiving data indicative of the level of a lysosomal system marker in whole blood treated with an inhibitor of lysosomal system function; and
a memory with software resident in the memory, and accessible to the processor, wherein the software comprises a series of instructions executable by the processor to convert the data to a measurement of lysosomal system flux in the subject.

In certain embodiments, the present disclosure provides a system for assessing lysosomal system flux in a subject, the system comprising:
a processor for receiving data indicative of the level of a lysosomal system marker in a sample of whole blood treated with an inhibitor of lysosomal system function; and
a memory with software resident in the memory, and accessible to the processor, wherein the software comprises a series of instructions executable by the processor to convert the data to a measurement of lysosomal system flux in the subject.

In certain embodiments, the present disclosure provides a system for assessing lysosomal system flux in a subject, the system comprising:
a processor for receiving data indicative of the level of a lysosomal system marker in a sample of whole blood and/or cells derived from a whole blood treated with an inhibitor of lysosomal system function; and
a memory with software resident in the memory, and accessible to the processor, wherein the software comprises a series of instructions executable by the processor to convert the data to a measurement of lysosomal system flux in the subject.

In certain embodiments, the system comprises a device. Devices are described herein.

In certain embodiments, the present disclosure provides a system for assessing lysosomal system flux in a subject, the system comprising:
a device for determining the level of a lysosomal system marker in whole blood, treated with an inhibitor of lysosomal system function;
a processor for receiving data from the device indicative of the level of the lysosomal system marker; and
a memory with software resident in the memory, and accessible to the processor, wherein the software comprises a series of instructions executable by the processor to convert the data to a measurement of lysosomal system flux in the subject.

In certain embodiments, the present disclosure provides a system for assessing lysosomal system flux in a subject, the system comprising:
a device for determining the level of a lysosomal system marker in a sample of whole blood, and/or in lysed cells derived from a whole blood, treated with an inhibitor of lysosomal system function;
a processor for receiving data from the device indicative of the level of the lysosomal system marker in the sample; and
a memory with software resident in the memory, and accessible to the processor, wherein the software comprises a series of instructions executable by the processor to convert the data to a measurement of lysosomal system flux in the subject.

In certain embodiments, the device for determining the level of a lysosomal system marker comprises a device for utilising immunological detection. Examples include ELISA based assays or flow cytometric techniques.

In certain embodiments, the system further comprise one or more other devices as described herein.

In certain embodiments, the device comprises a device for separating cells from whole blood. Examples of such devices include devices utilising density gradient centrifugation techniques or magnetic separation techniques.

Computer processors, and software for converting data to a measurement of a parameter, are known in the art.

Certain embodiments of the present disclosure provide methods of treating a subject suffering from, or susceptible to, a disease, condition or state associated with dysfunction of the lysosomal system.

In certain embodiments, the present disclosure provides a method of treating a subject suffering from, or susceptible to, a disease, condition or state associated with lysosomal system dysfunction, the method comprising determining the lysosomal system flux in the subject by a method as described and treating the subject on the basis of the level of lysosomal system flux determined.

Examples of conditions associated with lysosomal system dysfunction include obesity, diabetes, ageing, lysosomal storage diseases, cardiovascular diseases, Alzheimer's disease, Parkinson's disease, frontotemporal dementia, motor-neuron disease, and many cancers, all of which are likely to be caused by, or exacerbated by low lysosomal system flux. It is highly likely that other diseases of ageing are also exacerbated by low lysosomal system flux.

Methods for identifying subjects suffering from, or susceptible to, a disease, condition or state associated with lysosomal system dysfunction are known in the art. Methods of treatment of the diseases, conditions or states are also known in the art.

Certain embodiments of the present disclosure provide a method of identifying a marker present in blood indicative of lysosomal system flux.

In certain embodiments, the present disclosure provide a method of identifying a marker present in blood indicative of lysosomal system flux in a subject, the method comprising:
determining the level of a candidate marker indicative of lysosomal system flux in whole blood treated with an inhibitor of lysosomal system function; and
identifying the candidate marker as a marker indicative of lysosomal system flux.

Methods for determining the level of a candidate marker are as described herein. In certain embodiments, the candidate marker is a protein. In certain embodiments, the candidate marker is an RNA. In certain embodiments, the candidate marker is a small molecule. In certain embodiments, the candidate marker is lipid. Other types of markers are contemplated.

In certain embodiments, the marker present in blood is a plasma and/or serum marker.

In certain embodiments, the method comprises the use of blood from an animal and/or a human subject. In certain embodiments, the method comprises the use of a suitable animal model.

In certain embodiments, the marker is indicative of lysosomal system flux in the absence of treatment of blood with an inhibitor of lysosomal system function.

In certain embodiments, the marker is indicative of lysosomal system flux in whole blood treated with an inhibitor of lysosomal system function.

In certain embodiments, the method comprises machine learning, or more conventional computational analysis of ‘omic’ data-sets to identify the candidate marker as a marker indicative of lysosomal system flux. Methods for utilising machine learning and computational analysis are known in the art.

Standard techniques may be used for cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Molecular Cloning: A Laboratory Manual, 3rd ed., Vols 1, 2 and 3, J. F. Sambrook and D. W. Russell, ed., Cold Spring Harbor Laboratory Press, 2001; herein incorporated by reference.

The present disclosure is further described by the following examples. It is to be understood that the following description is for the purpose of describing particular embodiments only and is not intended to be limiting with respect to the above description.

### EXAMPLE 1

### Measurement of Autophagic Flux in Humans: An Optimised Method for Blood Samples

Abstract

Efficient autophagic flux is a critical cellular process that is vastly under-appreciated in terms of its importance to human health. Studies on mice have demonstrated that reductions in autophagic flux cause cancer and exacerbates chronic diseases including heart disease, and the pathological hallmarks of dementia. Autophagic flux can be increased by targeting nutrition or nutrition-related biochemical signaling. Currently this knowledge cannot be translated because there is no way to directly measure autophagic flux in humans. In this study we detail a method whereby human autophagic flux can be directly measured in peripheral blood mononuclear cells while still in whole human blood. Measurement of autophagic flux in cells while still in whole blood represents an important advance because it preserves genetic, nutritional, and signaling parameters inherent to the individual. This test is highly significant because it will allow the identification of factors that damage autophagic flux in humans, and also nutritional and pharmacological interventions that are capable of maintaining autophagic flux with ageing.

Introduction

Autophagic flux is the acquisition, transport, and degradation of unwanted or damaged material in the lysosomal system. The efficient execution of this entire series of events is important for two aspects of physiology. The first is cellular quality control that is critical for healthy tissue function. The second major function of autophagic flux is nutrient recycling to adapt to starvation. Accumulating human and preclinical research also shows inefficient autophagic flux plays a major and direct role in prevalent diseases such as dementia, and heart disease. Further, lysosomal system function supports healthy proteostasis, the dysfunction of which is a hallmark of aging. Consistent with this, mice that possess higher levels of autophagic activity actually live for longer.

As such, modification of lysosomal system function is important to human health and interventions that modify lysosomal system function (including nutrition, exercise, or pharmacological agents) should be translated into clinical practice. It is well known that caloric restriction increases autophagy, and more translatable nutritional interventions such as reduction of protein consumption may also promote this process. Aerobic exercise also increases autophagic function, and inhibitors of mTOR such as rapamycin, or compounds that augment sirtuin-1 activity, such as resveratrol, also augment autophagy.

Unfortunately, there are no methods that directly measure autophagic flux in human samples, and this is recognised as a barrier to translating interventions that target autophagy. Many studies have measured lysosomal system proteins in human samples, which has been used as a proxy for autophagic flux. This does not measure flux of material through the lysosomal system and is not a reliable measure of lysosomal system activity. The gold standard test for autophagic flux is western blot for an LC3 protein isoform without and with inhibition of lysosomal proteolysis. This technique is commonly applied to cells in culture. However, this method has not been adapted successfully to organotypic human samples that reflect both the nutritional and endocrine status of an individual; both factors directly impact mTOR signalling and thus lysosomal system function.

As such, it is not known what kinds of variation impact autophagic flux in a human population, or what important co-variates might look like. Further, because lysosomal flux has not been measured in humans, autophagic flux cannot be used a primary endpoint in itself for clinical trials. In the absence of such a measure, disease-specific endpoints would have to be used and the impact of treatments that aim to boost autophagic flux will remain unclear. This gap in knowledge represents an urgent unmet need that is currently hampering translation of a wealth of data on the lysosomal system that already exists in the scientific literature.

In this study, we present quantitative measurement of autophagic flux for the first time in an organotypic human sample. Here we present autophagic flux measured in peripheral blood mononuclear cells (PBMCs), while the cells still existed in whole human blood to preserve the individual-specific nutritional and cell signalling environment. This study tests different clinic-relevant factors that could cause measurement variation. We describe a protocol for measurement of autophagic flux that is easy to perform and well within the capability of most biochemical research laboratories. This test will be important for measuring the effect of lifestyle or pharmacological interventions in humans, for the first time, on the most biologically relevant lysosomal system parameter—autophagic flux.

Materials and Methods

Blood Collection and Whole-Blood Incubation

Blood was collected in lithium heparin (Greiner Bio-One, vacuette tube 9 ml lithium heparin, 455084), or EDTA (Greiner Bio-One, vacuette tube 9 ml K3EDTA, 455036) blood collection tubes (6-9 ml). Unless otherwise stated, blood samples were split into two (2×3 ml) and pipetted into 10 ml conical centrifuge tubes. One of the blood samples was treated with a final concentration of 150 μM of chloroquine (CQ, chloroquine diphosphate, Sigma Aldrich, C6628) (9 μl of 50 mM for a final volume of 3 ml of blood). Blood was mixed gently by inverting both tubes 3-4 times. Both tubes (+CQ and −CQ) were incubated at 37° C. for 1 hour with rotation (10 revolutions per minute, Thermo Scientific Tube Revolver, 88881002).

PBMC Isolation

After blood was incubated for 1 hour at 37° C., blood tubes were kept on ice to stop enzyme activity and vesicular trafficking. PBMCs were then isolated using standard procedures. To each tube, 3 ml of cold DPBS (Dulbecco's Phosphate-Buffered Saline, GIBCO) was added to 3 ml of blood (1:1) and samples were mixed by gently inverting tubes 3-4 times. Four ml of Lymphoprep (Stemcell Technologies, 07811) was carefully underlayed beneath the blood/DPBS mixture using a 10 mL syringe and a canula (sterile). This was repeated for both blood tubes (containing blood incubated without and with CQ). Tubes containing blood/DPBS underlayed with lymphoprep were centrifuged for 30 min at 800 g, with brake off at 4° C.

PBMCs were carefully aspirated with a 1 mL pipette and dispensed into a 10 mL conical centrifuge tube (approximately 2 ml volume containing PBMCs). PBMCs were then diluted with cold DPBS to a final volume of 5 ml. PBMCs and DPBS were mixed gently by inverting tubes 3-4 times. PBMCs were then pelleted by centrifugation at 600 g for 10 min at 4° C. (with brake). The supernatant was discarded.

Red Blood Cell Lysis

PBMCs were resuspended with 1 ml of red blood cell lysis buffer (1×, BD Biosciences, 555899) and were mixed by gently pipetting. PBMCs were left on ice for 2 minutes, after which they were pelleted by centrifugation at 600 g for 5 min at 4° C. The supernatant was discarded and PBMCs were washed by resuspension in 5 ml of cold DPBS. PBMCs were pelleted again by centrifugation at 600 g for 5 min at 4° C. The supernatant was discarded and the cells were again resuspended in 1 ml of cold DPBS and this suspension was transferred to a 1.5 ml microcentrifuge tube. This tube was centrifuged at 2,000 g for 10 min at 4° C. The supernatant was discarded and the pellet containing PBMCs was frozen on dry ice and stored at −80° C. until biochemical analyses.

Western Blotting

PBMC pellets were resuspended in a lysis buffer containing protease and phosphatase inhibitors (10 mM Tris pH 7.0, 1 mM EDTA, 0.5 mM EGTA, 1% Triton X-100, 0.1% sodium deoxycholate, 0.1% SDS, 140 mM NaCl, 2.5 mM sodium pyrophosphate, 1 mM sodium orthovanadate, 1 mM β-glycerophosphate (pH 7.4) protease inhibitor cocktail Roche (1×). Cell suspensions were then sonicated on ice 2×20 sec. Cell lysates were centrifuged at 2,000 g for 10 min at 4° C. and the supernatant collected.

Total protein was measured using a micro BCA protein assay kit [Thermo Fisher Scientific 23235]. Western blot analysis was performed using 10 μg of total protein of each of the homogenates that were electrophoresed at 130 V for 1 hour through 4-12% Bis-Tris Plus SDS-PAGE gels (Bolt™, Invitrogen, Thermofisher Scientific). The gels were transferred to a methanol-activated PVDF membrane at 35 V for 70 min. The membranes were then incubated in block solution (Tris-buffered saline containing 0.1% (v/v) Tween 20 (TBST) and 2% (w/v) bovine serum albumin (BSA) for 1 h at room temperature, with rocking.

The membranes were incubated overnight at 4° C. with primary antibodies diluted in block solution on a tube roller. Rabbit anti-MAP1LC3B (Novus Biologicals, NB100-2220, 1:1,000) was used to detect LC3B. The following day the membranes were washed three times for 5 min in TBST and then incubated for 1 h at room temperature, with rocking, with HRP-conjugated goat anti-rabbit (Merk Millipore, AP307P). The membranes were washed three times for 5 min in TBST and then developed using the WestFemto ECL blotting system (Thermo Scientific, 34095) and detected using the LAS4000 Luminescent Image Analyser (Fujifilm Life Science).

For a loading control we used HRP-conjugated anti-β-actin (Sigma-Aldrich, A3854) diluted 1:10,000 in block solution. Membranes were washed in TBST and incubated in block solution for 1 h at room temperature with rocking. The membranes were then incubated for 1 h at room temperature, with rocking, with HRP-conjugated anti-β-actin antibody solution. The membranes were washed three times for 5 min in TBST before being developed using the West Pico ECL blotting system (Thermo Scientific, 34077) and detected using the LAS4000 Luminescent Image Analyser (Fujifilm Life Science). Images were analysed using ImageJ software.

Results

Titration of Chloroquine for Measurement of Autophagic Flux in Whole Blood

The purpose of this experiment was to determine if lysosomal system flux could be measured in PBMCs while they were still in whole blood using chloroquine. This is important as autophagy in these cells will be influenced by the physiological status of the blood itself. Chloroquine was added directly to fresh whole blood across a concentration range, from 0 μM through to 200 μM. This titration was performed on blood from four different people. Cells were incubated at 37° C. for one hour while being rotated. At the end of that hour, PBMCs were isolated from the blood samples using Lymphoprep. PBMC samples were processed for western blotting and samples were probed for the autophagic cargo proteins LC3A, LC3B, LC3C, and P62, and the loading control proteins GAPDH, and β-actin.

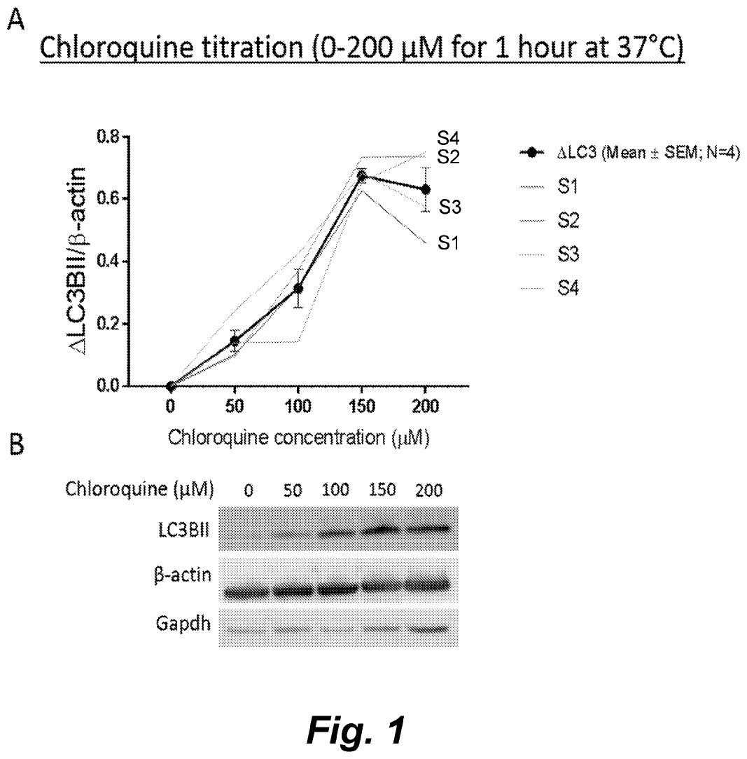

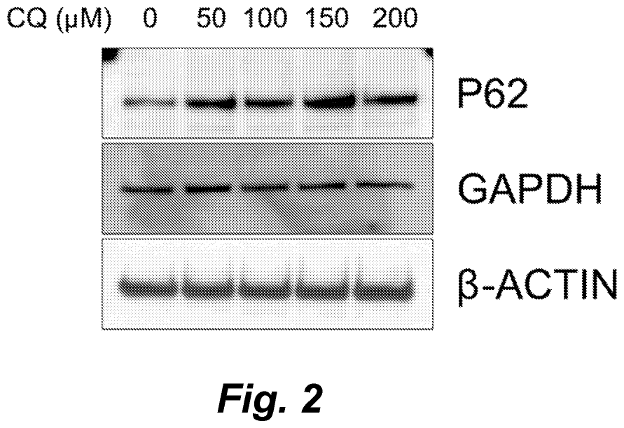

Out of these proteins, only LC3B was useful as a marker of autophagic flux in PBMCs that were exposed to chloroquine in whole blood (FIG. 1). LC3B-II displayed a robust directly proportional increase in response to increasing chloroquine concentration in whole blood up until 150 μM (FIG. 1). LC3B in FIG. 1 is quantified as fold increase over the no-chloroquine condition, and is designated ‘ΔLC3BII’, or a measure of autophagic flux. Even though LC3B was the most useful marker, P62 also showed increased abundance when chloroquine was added to blood, and that blood was incubated and processed in the same manner as for LC3B. Thus P62, and other LC3-interacting proteins could also be expoited as useful markers for measuring lysosomal system flux in blood (FIG. 2)

Incubating Blood with an mTOR Inhibitor Reveals Potential Autophagic Flux

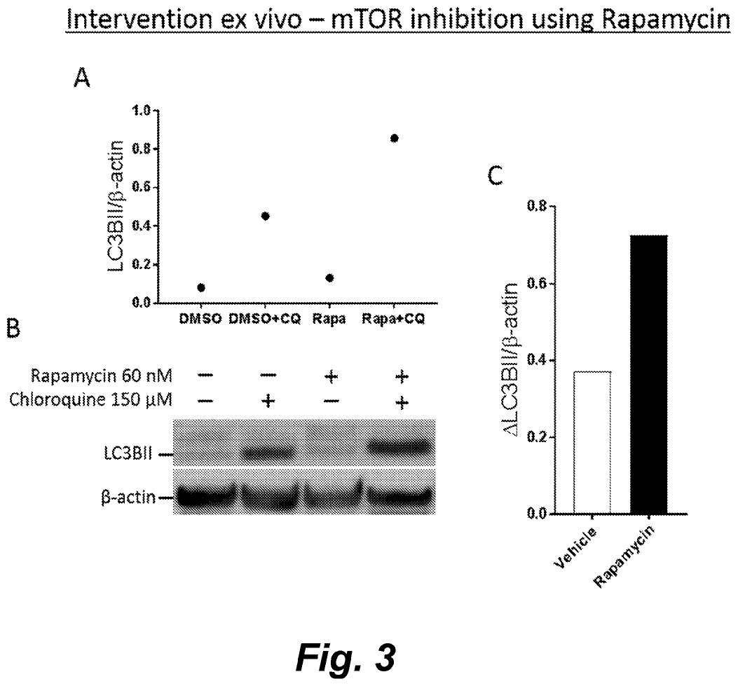

After determining that autophagic flux in PBMCs can be reliably measured by lysosomal inhibition in whole blood, we wanted to know whether the test was sensitive to changes in autophagic flux. To determine this, we exposed blood to rapamycin (a known inducer of autophagy) in order to increase autophagic flux (FIG. 3). Consistent with our hypothesis, autophagic flux, as measured by our test, increased in the presence of rapamycin.

Determining the Reproducibility of Autophagic Flux Measurements

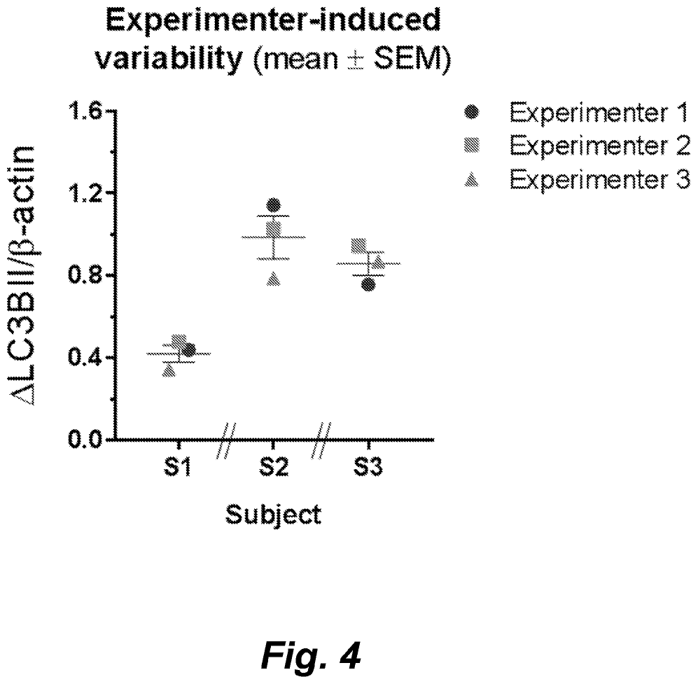

As autophagic flux is a dynamic process that responds to different kinds of cell stress, we were concerned about the reproducibility of measuring autophagic flux in human blood. To investigate the reproducibility of measurements made with human blood, we designed an experiment that would assess the variation caused by the experimenter conducting the measurement (FIG. 4).

To test variation caused by the experimenter, blood was collected from three different subjects and this blood from these three subjects was each split between three different experimenters. Samples were then processed, and flux was measured (FIG. 4). These data showed that variation within one subject measured by three different scientists was smaller than variation between different subjects.

Conclusions

Here we present an optimized protocol for the measurement of autophagic flux in organotypic samples taken from humans. Using human blood samples, we demonstrated that the amount of LC3BII in PBMCs increases in a manner that is directly proportional to the amount of chloroquine added to whole blood up to 150 μM. We further showed the method presented here displays low variation and responds in a predictable way to an ex-vivo treatment (rapamycin). Thus the method presented here is suitable for measurement of autophagic flux in humans in response to experimental interventions.

This tool will be central to the translation of data pertaining to the modification of autophagic flux for the prevention or treatment of disease. Several other studies have attempted to create a similar measure, but the technique presented here differs in critical ways. Firstly, whereas others have added lysosomal inhibitors to PBMCs isolated from human blood, we expose PBMCs to a lysosomal inhibitor (chloroquine) while still in whole blood. This is important as an individual's physiological characteristics that impact autophagy (such as blood glucose, amino acid concentrations, and circulating insulin) remain intact, important for faithful measurements of individual autophagic flux.

Finally, amounts of blood of 1 ml or less are suitable for analysis. In this regard, typically about 6 ml of blood is used, which is split in two 3 ml samples, one 3 ml sample without the inhibitor and one 3 ml sample with the inhibitor (eg chloroquine, bafolimycin). However, experiments have also been conducted that confirm that 500 μL samples of whole blood contain sufficient protein to conduct the assay.

### EXAMPLE 2

### Further Studies on Measurement of Autophagic Flux in Humans

Abstract

Efficient autophagic flux is a critical cellular process that is vastly under-appreciated in terms of its importance to human health. Studies on mice have demonstrated that reductions in autophagic flux cause cancer and exacerbates chronic diseases including heart disease, and the pathological hallmarks of dementia. Autophagic flux can be increased by targeting nutrition or nutrition-related biochemical signaling. Currently this knowledge cannot be translated because there is no way to directly measure autophagic flux in humans. In this study we detail a method whereby human autophagic flux can be directly measured in peripheral blood mononuclear cells while still in whole human blood. Measurement of autophagic flux in cells while still in whole blood represents an important advance because it preserves genetic, nutritional, and signaling parameters inherent to the individual. This test is highly significant because it will allow the identification of factors that damage autophagic flux in humans, and also nutritional and pharmacological interventions that are capable of maintaining autophagic flux with ageing.

Materials and Methods.

Blood Collection

Blood was collected in lithium heparin (Greiner Bio-One, Vacuette tube 9 mL lithium heparin; 455084), or EDTA (Greiner Bio-One, Vacuette tube 9 mL K₃EDTA; 455036) blood collection tubes (6-9 mL) from subjects who were fasted for a minimum of 12-hours. A total of 24 subjects provided blood in this study up to three-times over a six-month period. Samples were de-identified and appear simply as ‘subjects 1-24’ in this manuscript.

PBMC and PMN Isolation for Primary Culture

After blood collection, PBMCs and PMNs were isolated following standard procedures. To each blood sample, 8 mL of cold DPBS (GIBCO, Thermo Fisher Scientific; 14190250) was added to 8 mL of blood (1:1) and mixed by gently inverting 3-4 times. Twelve mL of Lymphoprep (Stemcell Technologies; 07811) was carefully underlaid beneath the blood/DPBS mixture using a 20 mL syringe and a canula (sterile) and centrifuged for 30 min at 800 g at room temperature, with brake off

PBMCs (white layer at the interface of plasma, upper phase, and Lymphoprep, translucent phase) were carefully aspirated with a 1 mL pipette and dispensed in a 10 mL conical centrifuge tube (approximatively 4 mL). PMNs (red bottom phase containing PMNs and erythrocytes) were collected with a 1 mL pipette and dispensed in a 10 mL conical centrifuge tube (2 mL). PBMCs and PMNs were then diluted with DPBS to a final volume of 10 mL and mixed gently by inverting 3-4 times, then pelleted by centrifugation at 600 g for 10 min (with brake). The supernatant was discarded.

PBMCs were resuspended with 1 mL of red blood cell lysis buffer (1×, BD Biosciences; 555899) and mixed by gently pipetting and incubated for 2 min, after which they were pelleted by centrifugation at 600 g for 5 min. PMNs were resuspended with 5 mL of red blood cell lysis buffer and mixed by gentle pipetting, incubated for 5 min, after which they were pelleted by centrifugation at 600 g for 5 min at 4° C. The supernatant was discarded and PMNs were washed by resuspension in 5 mL of DPBS. PMNs were pelleted again by centrifugation at 600 g for 5 min. The supernatant was discarded. This red blood cell lysis was repeated twice for the PMNs.

After red blood cell lysis, PBMCs and PMNs were washed twice in 5 mL DPBS and resuspended in 2.5 mL RPMI medium (Life Technologies; R8758) containing 10% fetal bovine serum (FBS) (Life Technologies; 10099-141); 0.5 mL of cell suspension was retained for cytometry analysis. Each sample was then split into two wells (1 mL/well in a 6-well plate) and each well was topped up with RPMI to a final volume of 3 mL/well. After one-hour incubation in a humidified incubator at 37° C., 5% CO₂ for each cell population, one well was treated with 150 μM chloroquine (3 μL/mL of media of 50 mM chloroquine solution diluted in sterile water) and the other with sterile water (3 μL/mL of media). Plates were incubated for one-hour in a humidified incubator at 37° C., 5% CO₂. After incubation, cells were harvested and centrifuged at 600 g for 5 min at 4° C. The pellets were washed by resuspension in 5 mL of cold DPBS. Cells were pelleted again by centrifugation at 600 g for 5 min at 4° C. and resuspended in 1 mL of cold DPBS and transferred to a 1.5 mL microcentrifuge tube. This tube was centrifuged at 2,000 g for 10 min at 4° C. The supernatant was discarded and the pellet containing PBMCs or PMNs was frozen on dry ice and stored at −80° C. for biochemical analyses.

Whole-Blood Incubation with Chloroquine

After collection, blood samples were split into two (3 mL each) and pipetted into 10 mL conical centrifuge tubes. One tube was treated with 150 μM final concentration chloroquine (CQ, chloroquine diphosphate; Sigma Aldrich; C6628) (9 μL/mL of 50 mM chloroquine solution diluted in sterile water). Blood was mixed gently by inverting both tubes 3-4 times. Both tubes (±CQ) were incubated at 37° C. for one-hour with rotation (10 revolutions/min; Thermo Fisher Scientific Tube Revolver; 88881002).

PBMC Isolation from Chloroquine-Treated Blood Samples

PBMCs were isolated using the following standard procedures. To each sample tube containing blood incubated without or with chloroquine, 3 mL of cold DPBS was added to 3 mL of blood (1:1) and samples were mixed by gently inverting tubes 3-4 times. Four mL of Lymphoprep was carefully underlaid beneath the blood/DPBS mixture using a 10 mL syringe and a canula (sterile) and tubes were centrifuged for 30 min at 800 g, with brake off at 4° C.

PBMCs (white layer at the interface of plasma, upper phase, and Lymphoprep, translucent phase) were carefully aspirated with a 1 mL pipette and dispensed in a 10 mL conical centrifuge tube (approximatively 2 mL). PBMCs were then diluted with cold DPBS to a final volume of 5 mL. PBMCs and DPBS were mixed gently by inverting tubes 3-4 times and pelleted by centrifugation at 600 g for 10 min at 4° C. (with brake). The supernatant was discarded.

PBMCs were resuspended with 1 mL of red blood cell lysis buffer and mixed by gentle pipetting, incubated on ice for 2 min, after which they were pelleted by centrifugation at 600 g for 5 min at 4° C. PBMC pellets were washed by resuspension in 5 mL of cold DPBS and pelleted again by centrifugation. The pelleted cells were resuspended in 1 mL of cold DPBS and transferred to a 1.5 mL microcentrifuge tube. This tube was centrifuged at 2,000 g for 10 min at 4° C. The supernatant was discarded, and the pellet containing PBMCs was frozen on dry ice and stored at −80° C. until analysis.

Generation of ATG5 KO Cells

HeLa cells were co-transfected with plasmids that expressed sgATG5/Cas9 (Addgene; LentiCRISPRv2-ATG5; Plasmid 99573) and GFP (Addgene; pUltrahot-GFP, modified from pUltrahot; Plasmid 24130) using Lipofectamine-2000 (Thermo Fisher Scientific; 11668027). After two-days, a single GFP-positive cell was plated/well in a 96-well plate using florescence-activated cell sorting (FACS). Monoclonal lines were amplified and screened for ATG5 KO and functional inhibition of autophagy by western blot using rabbit anti-ATG5 (1:1,000, Cell Signaling Technologies, D5F5U; 12994) and rabbit anti-LC3B (Novus; NB100-2220).

Ex Vivo Nutritional Intervention

Blood samples were split into four wells (4×3.5 mL) in a 6-well culture plate. Two wells were treated with 200 μM final concentration L-leucine (Sigma-Aldrich; L8912) (2 μL/mL of blood of 100 mM leucine) and an estimated final concentration of 400 nM of insulin (Sigma-Aldrich, 16634-50MG) (4 μL/mL of blood of 100 μM insulin solution). The two other wells were treated with sterile water (6 μL/mL of blood). Plates were incubated for 3 h in a humidified incubator at 37° C., 5% CO2. After incubation, leucine-+insulin-treated samples (2×3.5 mL) were pipetted into one 10 mL conical centrifuge tube and vehicle-treated samples (2×3.5 mL) into another 10 mL conical centrifuge tube. Samples were then split into two (2×3 mL) for each treatment. One sample for each treatment was treated with chloroquine, and the other was not treated with chloroquine as described above and incubated for 1 h at 37° C. with rotation before PBMCs were extracted for western blot analysis (see “PBMC isolation from chloroquine-treated blood samples” Material and method section).

Western Blotting

PBMC and PMN pellets were resuspended in a lysis buffer containing protease and phosphatase inhibitors (10 mM Tris, pH 7.0, 1 mM EDTA, 0.5 mM EGTA, 1% Triton X-100, 0.1% sodium deoxycholate, 0.1% SDS, 140 mM NaCl, 2.5 mM sodium pyrophosphate, 1 mM sodium orthovanadate, 1 mM β-glycerophosphate (pH 7.4), protease inhibitor cocktail Roche 1×). Cell suspensions were then sonicated on ice twice for 20 sec. Cell lysates were centrifuged at 2,000 g for 10 min at 4° C. and the supernatant collected. Total protein was measured using a micro BCA protein assay kit (Thermo Fisher Scientific; 23235).

Western blot analysis was performed using 10 μg of total protein of each of the homogenates that were electrophoresed at 130 V for 1 h through 4-12% Bis-Tris Plus SDS-PAGE gels (Bolt™; Invitrogen, Thermo Fisher Scientific). The gels were transferred to a methanol-activated PVDF membrane at 35 V for 70 min. The membranes were then incubated in block solution (Tris-buffered saline containing 0.1% (v/v) Tween 20 (TBST) and 2% (w/v) bovine serum albumin (BSA)) for 1 h at room temperature, with rocking.

The membranes were incubated overnight at 4° C. with primary antibodies diluted in block solution on a tube roller. The following primary antibodies were used: rabbit anti-MAP1LC3A (1:1,000, Abcam; ab62720), rabbit anti-MAP1LC3B (1:1,000, Novus Biologicals; NB100-2220), rabbit anti-MAP1LC3C (1:1,000, Cell Signaling Technology, clone D1R8V; 14723), mouse anti-SQSTM1/P62 (1:1000, Abnova, clone 2C11; H00008878-M01), rabbit anti-ATG5 (1:1,000, Cell Signaling Technologies, D5F5U; 12994).

The following day the membranes were washed three times for 5 min in TBST and then incubated for 1 h at room temperature, with rocking, with HRP-conjugated goat anti-rabbit (Merk Millipore; AP307P) or sheep anti-mouse (Merk Millipore; AC111P) diluted 1:10,000 in block solution. The membranes were washed three times for 5 min in TBST and then developed using the WestFemto ECL blotting system (Thermo Fisher Scientific; 34095) and detected using the LAS4000 Luminescent Image Analyzer (Fujifilm Life Science). Mouse anti-GAPDH antibody (Sigma-Aldrich; G8795) and HRP-conjugated anti-β-ACTIN (Sigma-Aldrich; A3854) diluted 1:10,000 in block solution were used as loading controls. For GAPDH, membranes were left to dry out then reactivated in methanol before being processed as described above. For β-ACTIN, membranes were washed in TBST and as above. The membranes were then incubated for 1 h at room temperature, with rocking, with HRP-conjugated anti-β-ACTIN antibody solution. The membranes were washed three times for 5 min in TBST before being developed using the West Pico ECL blotting system (Thermo Fisher Scientific; 34077) and detected on the LAS4000 Luminescent Image Analyzer. Images were analyzed using ImageJ software.

Flow Cytometry

Whole leukocytes, PBMCs and PMNs (n=3 of each) were analyzed by flow cytometry on the BD LSR Fortessa X20 Analyzer (BD Bioscience, USA) to confirm purity. Single cells were gated on FSC-H v FSC-A. Lymphocyte, monocyte and granulocyte populations were plotted based upon their size and granularity (FSC-A and SSC-A) using BD FACSDiva software version 8.0 (BD Bioscience, USA).

Statistical Analysis

Graphs and statistical analyses were generated using GRAPHPAD PRISM, version 8.2.0, for Windows (GraphPad Software, La Jolla, Calif., USA). Statistical analyses for ex vivo nutritional intervention were performed using a paired-t-test. Data in the figures are expressed as mean values with their standard errors (SEM). Results were considered significantly different when p<0.05.

Results

Primary Culture of Leukocytes Shows Autophagic Flux Can be Measured in Blood Cells In Vitro

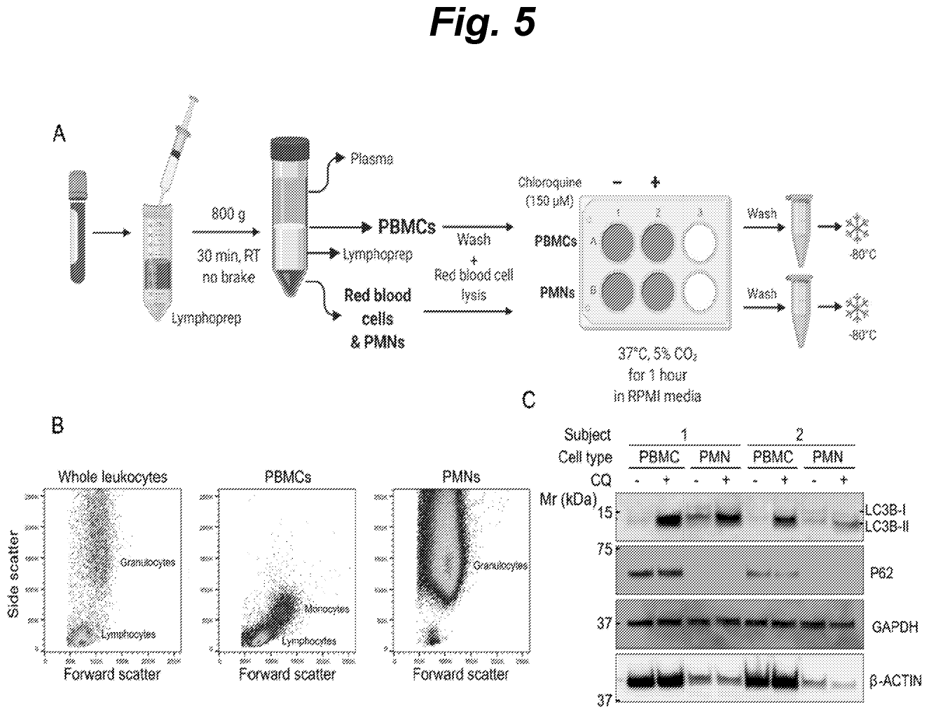

Blood from three individuals was subjected to two different kinds of cell isolation. Whole blood was fractionated using Lymphoprep to isolate either peripheral blood mononuclear cells (PBMCs) or polymorphonuclear cells (PMNs). After isolation, each cell sample was split into two and each cultured in media with or without 150 μM chloroquine for one-hour for analysis of autophagic flux (FIG. 5A). These populations were verified by flow cytometry (FIG. 5B). Autophagic flux was assessed by western blotting analysis for LC3B and SQSTM1/P62 (FIG. 5C). As expected, LC3B-II degradation was inhibited by chloroquine treatment, as observed by the increase in LC3B-II. Autophagic flux could therefore be investigated in these two cell populations. P62 did not change after chloroquine treatment in PBMCs, and was not detected in PMNs, making this cargo unsuitable for further investigation in these cell types (FIG. 5C). Given that PBMC isolation is much faster compared to PMN isolation, we pursued our experiments by using only PBMCs. Faster isolation of PBMCs compared with PMNs is important because more samples can be handled at once, and artefacts due to processing are less likely to occur.

Titration of Chloroquine in Whole Blood for Measurement of Autophagic Flux in PBMCs

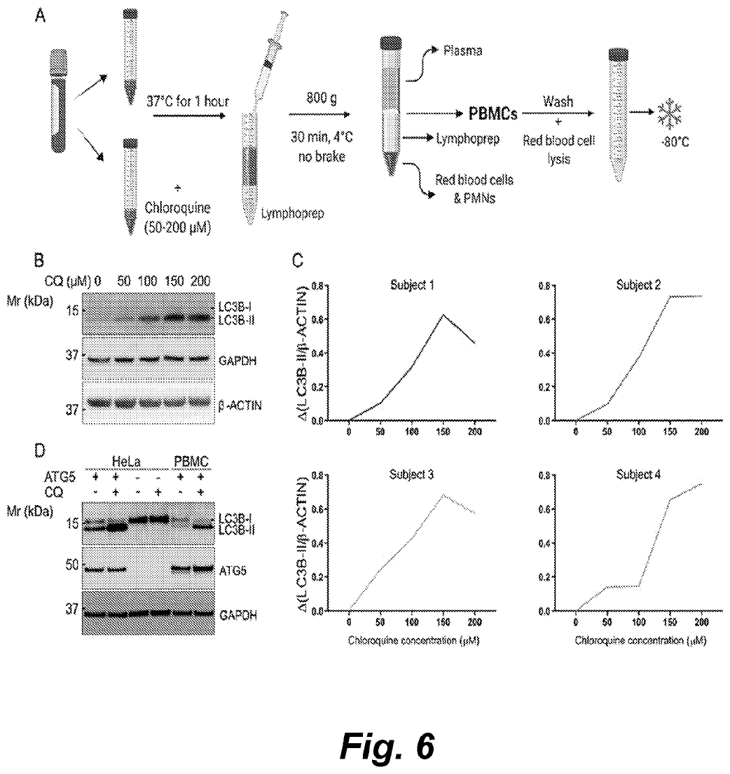

Having observed that measurement of autophagic flux was possible in primary cultures of PBMCs, we next determined whether chloroquine could be added to whole blood before PBMC isolation. The purpose of this experiment was to determine if flux could be blocked by chloroquine in PBMCs while they were still in whole blood, and then measured. This is important as autophagy in these cells will be influenced by the physiological status (e.g. nutrient content, insulin level) of the blood itself. Thus, chloroquine was added directly to fresh whole blood across a concentration range from 0 μM through to 200 μM. This titration was performed on blood from four subjects. Chloroquine treated blood samples were incubated at 37° C. for one-hour while being rotated. At the end of that hour, PBMCs were isolated from the blood using Lymphoprep (FIG. 6A). PBMC were processed for western blotting and samples were probed for the autophagic cargo proteins LC3A, LC3B, LC3C, and P62, and the loading control proteins GAPDH, and β-ACTIN (FIG. 6B, and data not shown).

While chloroquine clearly blocked autophagic flux in PBMCs when added to whole blood, not all markers correlated in a linear relationship with chloroquine concentration. Of the proteins analyzed, only LC3B-II was useful as a marker of autophagic flux in PBMCs that were exposed to chloroquine in whole blood (FIG. 2C). LC3B-II displayed a robust, directly proportional increase in response to increasing chloroquine concentration in whole blood up to 150 μM, after which a ceiling effect was observed (FIG. 6C). Autophagic flux in FIG. 6 is quantified as change in LC3B-II, displayed as:

Δ(LC3B-II/β-ACTIN)=(LC3B-II/β-ACTIN with chloroquine)−(LC3B-II/β-ACTIN without chloroquine)

LC3A was not clearly responding to chloroquine treatment and was unreliable between different subjects; LC3A-II was barely detectable in some samples, and LC3C was almost undetectable by western blot in PBMCs. Interestingly, P62 did not respond to chloroquine as well as LC3B, although increased protein abundance with chloroquine treatment was observed in three out of four subjects. Thus, we chose to treat whole blood with a final concentration of 150 μM chloroquine for one-hour and to analyze the change in LC3B-II as a measure of autophagic flux in PBMCs. Importantly, we noticed that GAPDH expression tended to co-vary with chloroquine treatment whereas β-ACTIN was more stable. Therefore, β-ACTIN was used to normalize LC3B-II values.

ATG5 KO cells that were generated by CRISPR-Cas9 genome editing were used to determine the specificity of the LC3B antibody that was used for this study. Whereas LC3B-II was clearly visible in wild-type HeLa cells, it was missing in ATG5 KO HeLa cells, and in line with a lipidation defect in the ATG5 KO cells; LC3B-I was more abundant. These bands were identical to bands detected in PBMCs (FIG. 6D).

Determining the Reproducibility of Autophagic Flux Measurements in Blood

As autophagic flux is a dynamic process that responds to different kinds of cell stress, we were concerned about the reproducibility of measuring autophagic flux in human blood. To investigate this we designed three experiments to test three different sources of variation—variation from the measurement system, variation from the biological system, and variation caused by the experimenter conducting the measurement.

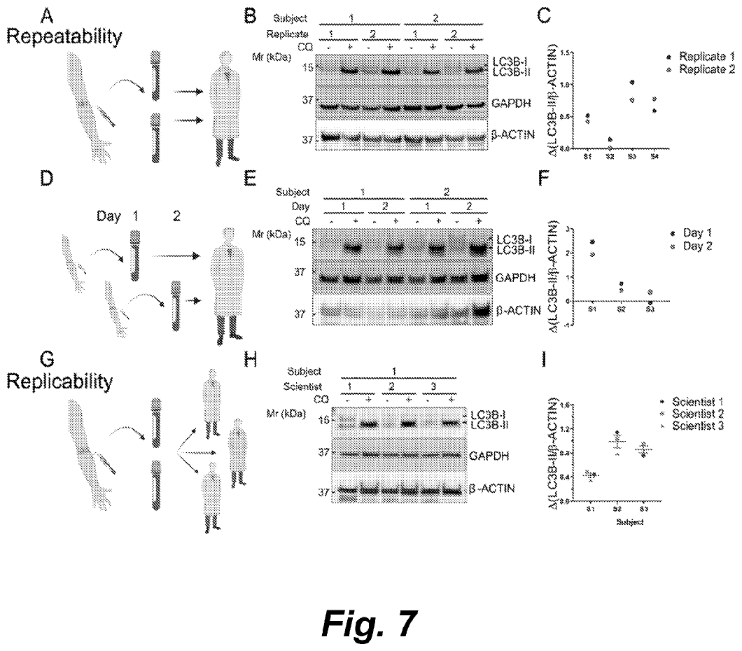

To test variation from the measurement system, we collected two vials of blood each from four subjects on the same occasion (FIG. 7A). The blood was processed and autophagic flux measured. Paired samples from the same subject were then compared to determine the reproducibility of the flux measurement (FIGS. 7B, C). Paired samples from the same subject processed in parallel on the same day gave similar autophagic flux values for both samples.

Variation from the biological system was measured by taking blood from the same subject (N=3) on two consecutive days under the same conditions (FIG. 7D). Blood samples were processed under the same conditions each day and autophagic flux was measured. Paired samples taken from the same subject on two consecutive days were compared (FIGS. 7D, F). We observed good reproducibility for autophagic flux measurements.

To test variation caused by the experimenter, blood was collected from three subjects on the same day and provided to three scientists for independent processing and analysis of autophagic flux (FIG. 7G). The results were similar for each subject (FIGS. 7H, I). Intra-individual variability of the autophagic flux measurement was low whereas we could detect inter-individual variability allowing further experiments to evaluate factors that impact individual autophagic activity.

Autophagic Flux is Affected by Short-Term Storage of Blood in the Laboratory

To determine the impact of short-term processing delays on autophagic flux measurement, we took two vials of whole blood each from three subjects. One vial was processed immediately after collection (delay <30 minutes) and the other vial was kept at room temperature for four-hours. This experiment was also conducted on blood collected from three additional subjects to investigate the impact of four-hours of storage of whole blood at 4° C. Blood was processed as described above: one-hour incubation at 37° C. with/without chloroquine followed by PBMC isolation. LC3B was analyzed by western blot.

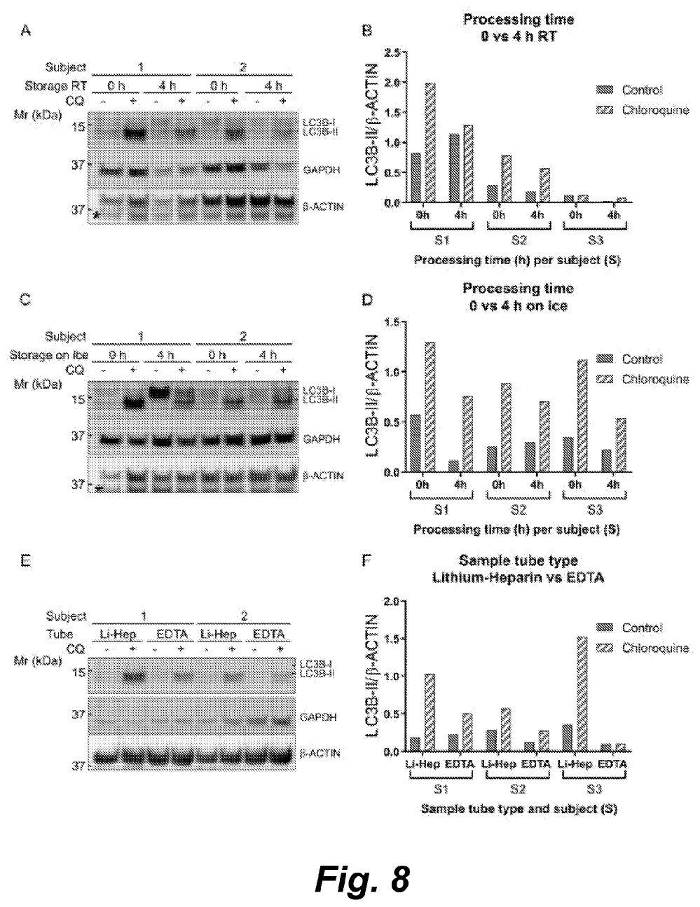

We observed different responses between blood that was immediately processed or held at room temperature for four-hours before analysis (FIGS. 8A, B). Although there were no consistent changes after storage between the three subjects analyzed, large changes in flux were observed that exceeded variability observed in the experiments shown in FIG. 3. Blood that was kept on ice for four-hours before processing also appeared to change with respect to LC3B-II-based flux analysis but was more stable than blood kept at room temperature for the same length of time (FIGS. 8C, D).

This experiment showed it is better to process blood as soon as possible after collection as autophagic activity does change over a period of hours, and it does so somewhat unpredictably. However, maintaining blood on ice can preserve the flux to a certain extent. In any case, it is critical to process blood consistently and within the shortest amount of time as possible.

Autophagic Flux is Differentially Affected by Blood Collection Tubes Containing Lithium-Heparin or EDTA

As blood can be collected in tubes containing different anti-coagulation factors, it was important to determine whether this impacted autophagic flux measurement. To test this, we collected blood from three subjects in tubes either containing EDTA or lithium-heparin as anti-coagulation reagents. Whole blood was treated with or without 150 μM chloroquine and incubated for one-hour at 37° C. before isolation of the PBMCs. LC3B-II was analyzed by western blot. We found autophagic flux (ΔLC3B-II) was greater in samples collected in tubes with lithium-heparin compared with EDTA. LC3B-II signal in no-chloroquine samples (basal state) was also lower in EDTA tubes, suggesting that EDTA may reduce either autophagy or LC3B-II readout (FIGS. 8E, F). Conversely, lithium-heparin may also be increasing autophagic flux. Based on these data, lithium-heparin tubes were used to complete experiments because autophagic flux was more reliably detected in blood taken using these tubes.

Autophagic Flux Measurement in Blood Allows Detection of Changes Induced by Nutrient Signaling

After determining that autophagic flux in PBMCs can be reliably measured by lysosomal inhibition in whole blood, we determined whether we could measure a predicted outcome with an autophagy-relevant intervention. Autophagy is highly responsive to nutrition and subject to regulation via the mTOR pathway. In the presence of amino acids, particularly leucine and insulin, mTORC1 is activated which in turn inhibits autophagy. Thus, we pre-treated blood ex vivo with physiological concentrations of L-leucine and insulin observed in plasma after whey-protein intake.

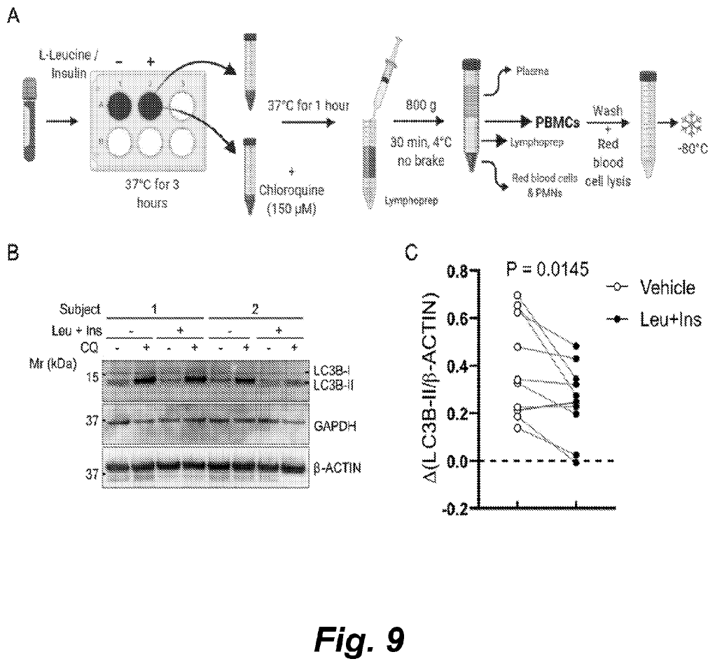

Blood was collected from 10 subjects, and cultured with or without a combination of L-leucine (200 μM) and insulin (400 nM) for three-hours at 37° C. before addition of chloroquine for one-hour (FIG. 9A). As expected, we observed a significant decrease in ΔLC3B-II between non-treated and leucine-+insulin-treated samples (P=0.0145, paired t-test) indicating a decrease in autophagic flux. However, two blood samples out of the 10 did not respond to this treatment, with flux remaining stable or slightly increasing. The result confirms this method of autophagic flux measurement can detect differences or changes in autophagy in human blood.

Discussion

Here we present an optimized protocol to measure autophagic flux in organotypic human blood samples. This is opposed to cell studies in vitro which do are reflect the nutritional and signaling status in an individual. Importantly, this method reports autophagic flux in a system with endocrine signaling, nutritional status, and genetic make-up inherent to the individual. Using human blood, we demonstrated that the amount of LC3B-II in PBMCs increases in a manner that is directly proportional to the amount of chloroquine added to whole blood up to 150 μM. We further showed that the method displays low variation and responds in a predictable way to an ex vivo treatment (leucine+insulin). Thus, the method is suitable for measurement of autophagic flux in humans in response to experimental interventions, or for determining the impact of important parameters such as age, body composition, or disease status.

Several other studies have attempted to create a similar measure but the technique presented here differs in critical ways. Firstly, whereas others have added lysosomal inhibitors to PBMCs isolated from human blood, we expose PBMCs to a lysosomal inhibitor (chloroquine) while still in whole blood. This is important as an individual's physiological characteristics that impact autophagy (such as blood glucose, amino acid concentrations, and circulating insulin) remain intact, important for faithful measurement of individual autophagic flux. Furthermore, one study attempted to inhibit autophagic flux in primary culture of PBMCs isolated from human blood using leupeptin as a lysosomal inhibitor in vitro, but failed to demonstrate autophagic flux measurement.

Our experiments to investigate variability in autophagic flux in human blood showed that intra-individual variation remained low whereas inter-individual or ex vivo treatment-induced variations were detectable. This means that adding chloroquine to whole blood can be used to measure inter-individual or inter-treatment comparisons. In vivo, we expect variation between individuals to be caused by both genetic and environmental factors. Overall system performance, measured by autophagic flux, is the product of hundreds of different genes. These genes display significant heterogeneity, which also shows association with Alzheimer's and Parkinson's diseases

Environmental factors will also alter flux, as also measured in this study. Participants in this study were fasted before blood was taken, which will produce a measurement with less day-to-day variation (as measured in FIG. 3D-F). However, other factors such as obesity, exercise, and treatment with drugs such as metformin should also alter results. This was directly demonstrated by adding physiological concentrations of leucine and insulin to whole blood, and the observation that this decreased autophagic flux (FIG. 9). This occurs because amino acids, particularly leucine, work with insulin to activate mTORC1, and this will inhibit autophagy.

The method presented here has some limitations. Although it allows measurement of autophagic flux in organotypic human samples, it must be performed immediately on fresh blood: storage at room temperature or on ice for four-hours led to increased variation in the abundance of LC3B-II in both basal and chloroquine-treated samples. Future studies should investigate biomarkers that co-vary with flux that can be detected in frozen plasma samples, for example.

Anti-coagulant agents used in blood collection also influence autophagy. That is true for both lithium (although millimolar concentrations are required for this effect) and EDTA, both of which were tested in this study. This means that the type of blood collection tube used for a study must be consistently used across groups as not to introduce experimental artefacts. Further, quantitation by western blot in general is both too variable and low-throughput to scale up to very large cohorts. Adapting this method for biochemical analysis in an ELISA plate format to detect LC3B-II will be important for large studies. Finally, whether autophagic flux in PBMCs correlates well with flux in other tissues also requires investigation under a variety of conditions.

In conclusion, we show that adding chloroquine to whole blood permits measurement of autophagic flux in PBMCs while still experiencing physiologically relevant cues that are faithful to the environmental factors experienced by that individual. This test will be useful for measurement of important factors that are likely to impact autophagic flux such as age, obesity, and diseases such as diabetes and Alzheimer's. This is the first such demonstration of autophagic flux in a human biological sample and development of this method will permit use of autophagy as an endpoint in clinical trials in and of itself.

### EXAMPLE 3

### Effect of Chloroquine or Bafilomycin Added to Whole Blood on LC3BII Accumulation

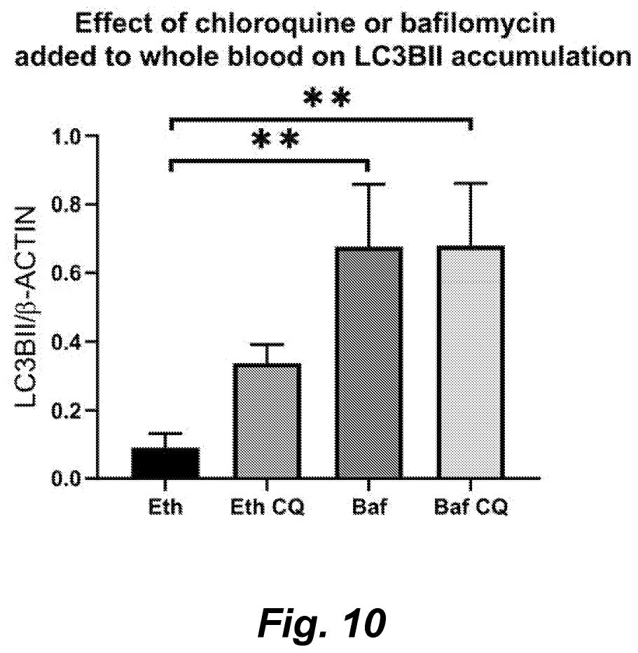

FIG. 10 shows that bafilomycin in an ethanol vehicle is also capable of stopping autophagic flux when added to whole blood.

Whole blood from four individuals was incubated with ethanol vehicle (Eth), chloroquine and ethanol vehicle, bafilomycin in an ethanol vehicle, or with both bafilomycin in an ethanol vehicle and chloroquine for one hour at 37° C. PBMCs were then isolated and analysed for LC3BII using western blotting.

Bafilomycin increased LC3BII cargo in these PBMC samples and when added together, CQ and bafilomycin did not have an additive effect, indicating that chloroquine blocks autophagic flux, and does not induce autophagy in these conditions. Bars=mean±SEM. **=P<0.01.

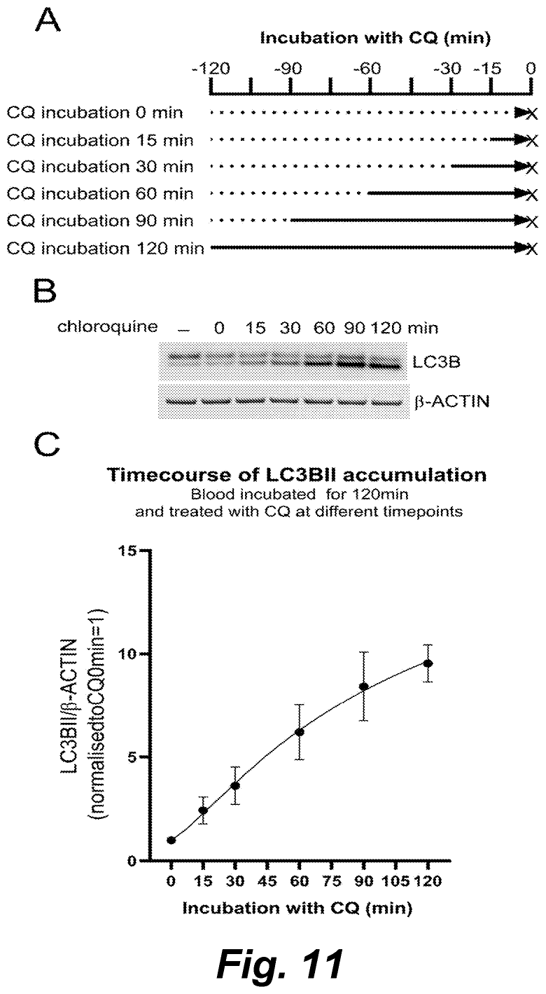

FIG. 11 shows a time course analysis of chloroquine incubation in whole blood. (A) 150 μM chloroquine was incubated with whole blood for the times indicated before harvest (X). (B) PBMCs were then isolated and analysed for LC3BII by western blotting. (C) Quantitative analysis of western blots revealed a near-linear increase in LC3BII accumulation over the times indicated (0-120 minutes of incubation).

It will be appreciated that the current studies may also be used to provide software executable by a computer processor to determine the lysosomal flux using an algorithm to convert the level of a marker of lysosomal flux determined to a parameter indicative of the lysosomal flux. Software utilising an algorithms may be developed to assess lysosomal flux.

The current studies described herein also provide support for methods and reagents for use in a kit for assessing lysosomal flux. A kit may contain one or more reagents as described herein and/or instructions for using the kit.

In addition, the current studies provided herein also provide support for screening methods to identify new markers for assessing lysosomal flux. In this case, candidate markers (eg proteins, RNAs, or other molecules) can be screened to determine whether the candidate markers reflect lysosomal system flux as determined by utilising a method as described herein for assessing lysosomal system flux.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “about” or “approximately” means an acceptable error for a particular value, which depends in part on how the value is measured or determined. In certain embodiments, “about” can mean one or more standard deviations. When the antecedent term “about” is applied to a recited range or value it denotes an approximation within the deviation in the range or value known or expected in the art from the measurements method. For removal of doubt, it shall be understood that any range or value stated herein that does not specifically recite the term “about” before the range or before any value within the stated range inherently includes such term to encompass the approximation within the deviation noted above.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of assessing lysosomal system flux in a subject, the method comprising determining the level of a lysosomal system marker in a sample of whole blood from the subject, the level of the lysosomal system marker being determined based on the level of the marker following treatment of the whole blood with an inhibitor of lysosomal system function.

2. The method according to claim 1, wherein the inhibitor of lysosomal system function comprises one or more of chloroquine, bafilomycin A1, E-64d, leupeptin, and pepstatin A.

3. The method according to claim 1, wherein the method comprises determining the level of the lysosomal system marker in one or more of peripheral blood mononuclear cells, polymorphonuclear cells, and other cellular populations, derived from the whole blood treated with the inhibitor of lysosomal system function.

4. The method according to claim 1, wherein the lysosomal system marker comprises an LC3 and/or a GABARAP/GATE-16 protein and/or an LC3 interacting cargo adaptor protein.

5. The method according to claim 1, wherein the lysosomal system marker comprises a LC3B protein.

6. The method according to claim 1, wherein the lysosomal system marker comprises LC3B-II protein or a P62 protein.

7. The method according to claim 1, wherein the method comprises comparison of the level of the lysosomal system marker with the level of a marker in whole blood without treatment with an inhibitor of lysosomal system function.

8. The method according to claim 1, wherein the method comprises determining the level of the lysosomal system marker using immunological detection.

9. The method according to claim 8, wherein the immunological detection comprises ELISA or immunocytochemical staining.

10. The method according to claim 8, wherein the immunological detection comprises Western blotting.

11. The method according to claim 1, wherein the method comprises:
obtaining a sample of whole blood from the subject;
treating the sample of whole blood with the inhibitor of lysosomal system function; and
determining the level of the lysosomal system marker in the whole blood so treated as compared to the level of the lysosomal system marker in whole blood without treatment.

12. (canceled)

13. A kit for assessing lysosomal system flux in whole blood using the method according to claim 1, the kit comprising the following components:
a reagent for detecting the lysosomal system marker; and
optionally one or more of an inhibitor of lysosomal system function, an anti-coagulant,
a biochemical extract reagent and a cell lysis reagent.

14. A system for assessing lysosomal system flux in a subject, the system comprising:
a processor for receiving data indicative of the level of a lysosomal system marker in whole blood treated with an inhibitor of lysosomal system function; and
a memory with software resident in the memory, and accessible to the processor, wherein the software comprises a series of instructions executable by the processor to convert the data to a measurement of lysosomal system flux in the subject.

15. The system according to claim 14, wherein the system further comprises a device for determining the level of a lysosomal system marker in a sample of cells and/or lysed cells derived from a whole blood treated with an inhibitor of lysosomal system function.

16. A method of treating a subject suffering from, or susceptible to, a disease, condition or state associated with autophagic dysfunction, the method comprising determining the lysosomal system flux in the subject by a method according to claim 1, and treating the subject on the basis of the level of lysosomal system flux determined.

17. A method of identifying a marker present in blood indicative of lysosomal system flux in a subject, the method comprising:
determining the level of a candidate marker indicative of lysosomal system flux in whole blood treated with an inhibitor of lysosomal system function; and
identifying the candidate marker as a marker indicative of lysosomal system flux.

18. The method according to claim 17, wherein the marker present in blood is a plasma and/or serum marker.

19. The method according to claim 17, wherein the marker is indicative of lysosomal system flux in the absence of treatment of whole blood with an inhibitor of lysosomal system function.

20. The method according to claim 17, wherein the method comprises computational analysis and/or machine learning to identify the candidate marker as a marker indicative of lysosomal system flux.
